(19) United States
(12) Patent Application Publication
(10) Pub. No.: US 2020/0187573 A1
(43) Pub. Date: Jun. 18, 2020

(54) Heat Therapy Leggings

(71) Applicant: Brittany Catherine Junod, Manhattan Beach, CA (US)

(72) Inventor: Brittany Catherine Junod, Manhattan Beach, CA (US)

(21) Appl. No.: 16/222,215

(22) Filed: Dec. 17, 2018

(51) Int. Cl. A41D 1/06; A41D 27/20; A61F 7/02

(52) U.S. Cl. CPC A41D 1/06; A41D 27/201; A61F 7/02; A61F 2007/0022; A41D 2400/10; A61F 2007/0236; A61F 2007/0238; A41D 2400/32

(57) ABSTRACT

Heat therapy leggings are described herein. The heat therapy leggings may include a double walled waistband. The double walled waistband may include an interior pocket to cover the front torso/midsection or lower back. The heat therapy leggings may be front to back reversible. A heat patch may be inserted into the interior pocket to provide relief from menstrual cramps.

### FIELD OF INVENTION

This application is in the field of heat therapy and leggings.

### BACKGROUND

Women have many lower back issues. One of the main causes of lower back pain is due to menstrual cramps. Additionally, menstrual cramps may be felt in the lower abdomen. Heat may be used to help relieve the pain of menstrual cramps.

Heat is proven to reduce menstrual cramps better than any medication, for example, ibuprofen or the like. One way to apply heat may be to use heat patches. These heat patches, however, may have several issues. For example, the heat patches may get too hot when directly applied to the skin, they do not stick correctly, or they fall off. One way to solve these issues may be to place these heat patches in a pocket.

Women wear leggings for a variety of reason, for example, leisure, exercise, or the like. Leggings may come in a variety of materials, for example, cotton, rayon, lycra, and the like. Leggings may come in a variety of shapes and sizes, for example, long, short, cropped, flared bottomed, high-waisted, and the like.

Legging may have a pocket. The pocket is generally small in size. For example, the pocket may hold a small cell phone or keys. The small cell phone fits snugly into the pocket with no extra rom. The pocket may be located in either the waist or the leg.

Leggings are worn by women at all times of the day, month, and year. There is a need to combine leggings with a heat therapy to aid in women's menstrual discomfort.

### SUMMARY OF THE INVENTION

Heat therapy leggings are described herein. The heat therapy leggings may include a double walled waistband. The double walled waistband may include an interior pocket to cover the front torso/midsection or lower back. The heat therapy leggings may be reversible.

### BRIEF DESCRIPTION OF THE DRAWINGS

FIG. 1 is an example of heat therapy leggings;

FIG. 2 is an example of a heat patch inside a pocket of the waistband; and

FIGS. 3A-3C are an example the pocket; and

FIG. 4 is an example of a pocket inside the heat therapy leggings.

### DETAILED DESCRIPTION OF THE DRAWINGS

This invention is described in the following description with reference to the Figures, in which like reference numbers represent the same or similar elements. While this invention is described in terms of modes for achieving this invention's objectives, it will be appreciated by those skilled in the art that variations may be accomplished in view of these teachings without deviating from the spirit or scope of the present invention. The embodiments and variations of the invention described herein, and/or shown in the drawings, are presented by way of example only and are not limiting as to the scope of the invention.

Unless otherwise specifically stated, individual aspects and components of the invention may be omitted or modified, or may have substituted therefore known equivalents, or as yet unknown substitutes such as may be developed in the future or such as may be found to be acceptable substitutes in the future. The invention may also be modified for a variety of applications while remaining within the spirit and scope of the claimed invention, since the range of potential applications is great, and since it is intended that the present invention be adaptable to many such variations.

Heat therapy leggings may be disclosed herein. The heat therapy leggings may include a double walled waistband. The double walled waistband may include an interior pocket to cover the front torso/midsection or lower back. The interior pocket may be large enough fit a heating patch for relief from menstrual cramps.

The heat therapy leggings described herein may be reversible. In this instance, reversible means either forwards or backwards. For example the interior pocket may be worn in either the front or back to relieve lower back pain or menstrual cramps.

The heat therapy leggings described herein may have an interior pocket the same size as a heat patch. The heat patch may be placed inside this interior pocket to keep the heat patch in place and prevent the skin from getting too hot. For example, the interior pocket may be 9×5 inches or 8×5 inches, large enough to fit a heat therapy patch.

The heat therapy leggings described herein may be high-waisted. The high waistband of the leggings may allow for the pocket to sit at either the lower back or front torso/midsection of the wearer. By having the interior pocket sit at either the lower back or torso/midsection of the wearer, the heat patch may apply heat in the area of discomfort.

The heat therapy leggings described herein may be made of any material, for example, cotton, rayon, lycra, and the like. The heat therapy leggings described herein may be any length or style, for example, long, short, cropped, flared bottomed, and the like.

The interior pocket may have an opening at or near the waistband of the heat therapy leggings. For example, the opening may be an open slot or require the use of a zipper to open and close. The opening may allow for insertion of the heat patch.

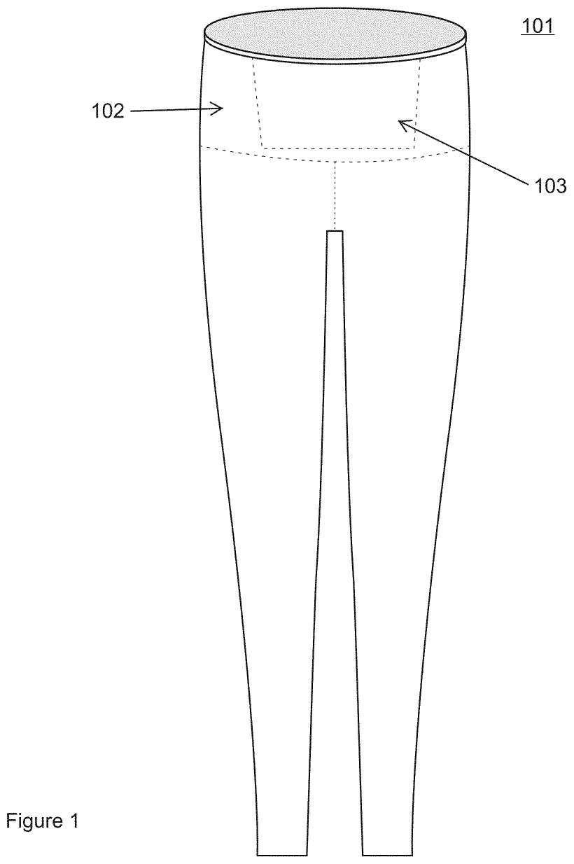

FIG. 1 is an example of the heat therapy leggings. The heat therapy leggings 101 have a high waist 102. The high waist 102 may include a pocket 103. The pocket 103 may be the same size and shape of a heat patch (not shown). The heat patch may be placed inside pocket 103 to provide relief of menstrual cramps.

The heat therapy leggings 101 may be reversible. For example, the pocket 103 may be in either the front or the back of the heat therapy leggings 101.

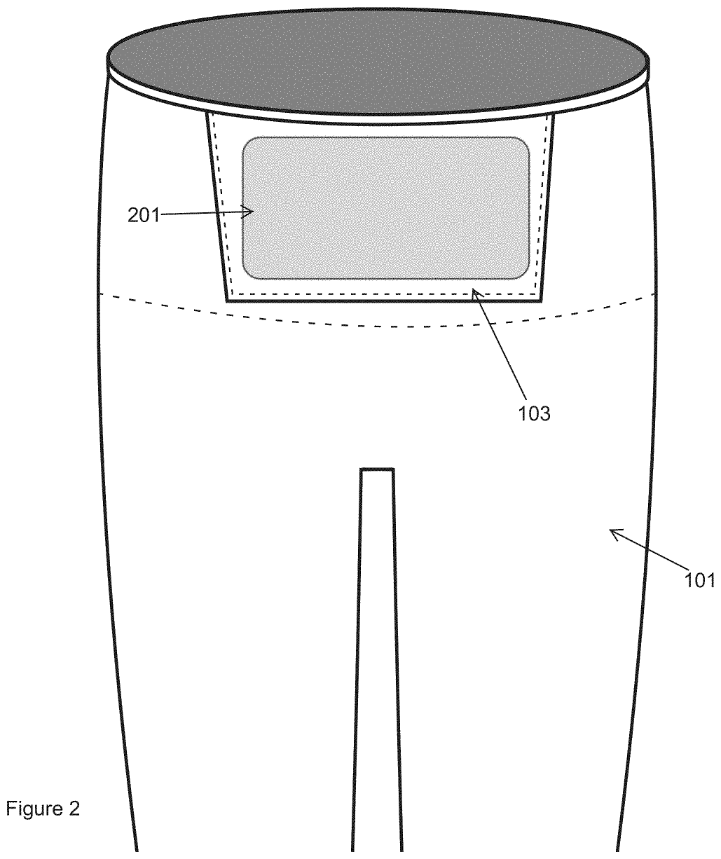

FIG. 2 is an example of a heat patch inside a pocket of the waistband. The heat patch 201 may be placed inside the pocket 103 of the heat therapy leggings 101 by a user. The user may open the pocket 103 and place a heat patch 201 inside. The heat patch 201 may provide relief from menstrual cramps. The pocket 103 may help keep the heat patch 201 in place and reduce the direct skin contact. When the heat patch 201 comes in direct contact with the skin it may be too hot for the user.

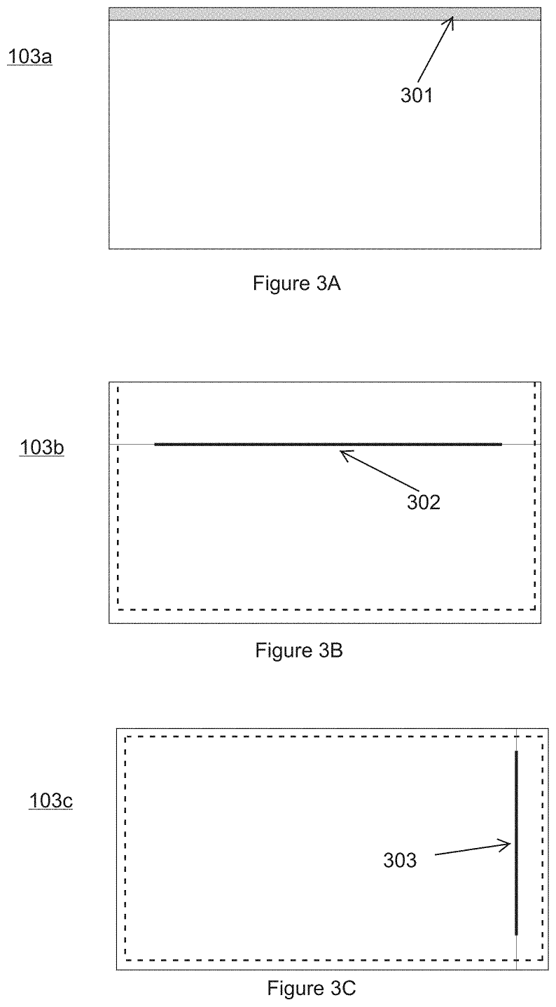

FIGS. 3A-3C are an example the pocket. The pocket 103 may be rectangular. In FIG. 3A, the pocket 103a may open at the top 301. In this example, a user may open the top of top 301 of the pocket 103a and slide in a heat patch.

FIG. 3B illustrates a pocket with a fold over split opening. The pocket 103b may have a fold over split opening 302. In this example, a user may open the pocket 103b at the split opening 302 (which may be somewhere in the middle of pocket 103b) and insert a heat patch.

FIG. 3C illustrates a pocket with a side slit opening. The pocket 103C may have a side slit opening 303, for example, on the right side of the pocket 103c as shown. In this example a user may open the pocket 103c at the side slit opening 303 and insert a heat patch. The side slit opening 303 may also be on the left side of the pocket 103c.

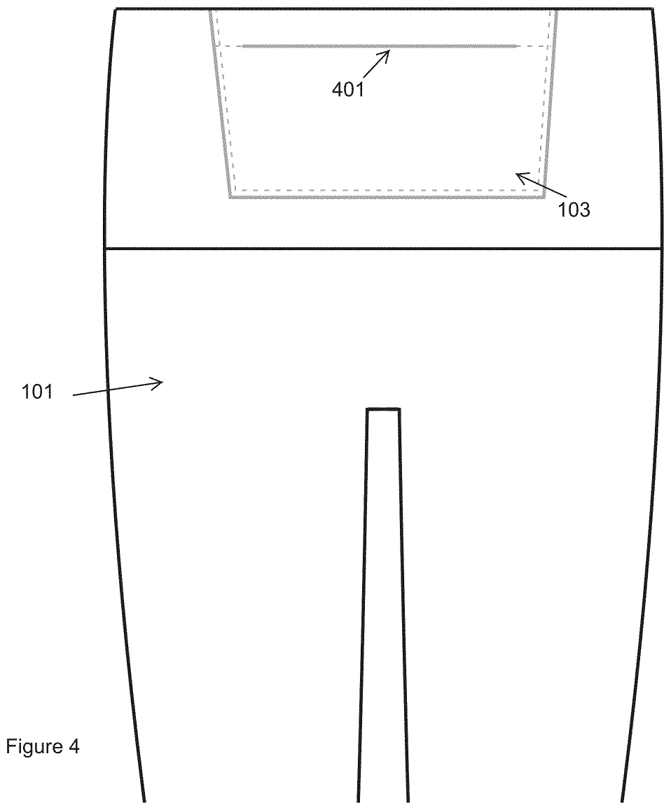

FIG. 4 is an example of a pocket inside the heat therapy leggings. The heat therapy leggings 101 may include a pocket 103. The heat therapy leggings 101 may have a double wall waist band, while the legs may be single wall. The pocket may be between the double wall waist band. The pocket 101 includes an opening 401 for inserting a heat patch.

It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments.

While multiple embodiments are disclosed, still other embodiments of the present invention will become apparent to those skilled in the art from this detailed description. The invention is capable of myriad modifications in various obvious aspects, all without departing from the spirit and scope of the present invention. Accordingly, the drawings and descriptions are to be regarded as illustrative in nature and not restrictive.

What is claimed:

1. Heat therapy leggings comprising:
a double wall waistband; and
an interior pocket, wherein the interior pocket is within the double wall waistband;
wherein a heat patch is placed inside the interior pocket.

2. The heat therapy leggings of claim 1, wherein the heat therapy leggings are reversible.

3. The heat therapy leggings of claim 1, wherein the interior pocket sits at the torso of a wearer.

4. The heat therapy leggings of claim 1, wherein the interior pocket sits at the lower back of a wearer.

5. The heat therapy leggings of claim 1, wherein the interior pocket sits at the midsection of a wearer.

6. The heat therapy leggings of claim 1, wherein the interior pocket opens at a top of the interior pocket.

7. The heat therapy leggings of claim 1, wherein the interior pocket has a fold over split opening.

8. The heat therapy legging of claim 1, wherein the interior pocket has a side slit opening.

9. The heat therapy leggings of claim 1, wherein the legs of the heat therapy leggings are single wall.

10. The heat therapy leggings of claim 1, wherein the interior pocket is the size of the heat patch.

11. The heat therapy leggings of claim 1, wherein the interior pocket is 9×5 inches.
